(19) United States
(12) Patent Application Publication
(10) Pub. No.: US 2015/0142329 A1
(43) Pub. Date: May 21, 2015

(54) METHOD AND SYSTEM FOR EXERCISE MONITORING

(71) Applicant: RLab Ab Oy, Vaasa (FI)

(72) Inventors: Fredrik ÖSTMAN, Vaasa (FI); Jonatan RÖSGREN, Vaasa (FI)

(73) Assignee: RLab Ab Oy, Vaasa (FI)

(21) Appl. No.: 14/546,501

(22) Filed: Nov. 18, 2014

(30) Foreign Application Priority Data
Nov. 19, 2013 (FI) 20136155
Sep. 15, 2014 (FI) 20145805

(51) Int. Cl. G01P 15/18; A61B 5/00; A61B 5/0205; A61B 5/11

(52) U.S. Cl. CPC G01P 15/18; A61B 5/1118; A61B 5/4866; A61B 5/0205; A61B 5/024
USPC 702/19; 702/141

(57) ABSTRACT

A portable data processing system includes a memory system, a processing system and a reception system for receiving data from acceleration sensors indicating test subject's acceleration in three orthogonal directions plus orientation of the acceleration sensors. The memory system includes routines for 1) determining instantaneous vertical, longitudinal and lateral accelerations from the received acceleration sensor data; 2) identifying periodicity in one of the instantaneous accelerations and using the periodicity with the instantaneous accelerations to determine dynamic movement parameters related to movement over ground, separately for both legs; 3) using a vertical energy loss and brake energy loss to determine a combined energy loss; and 4) outputting an indication of the combined energy loss and/or correction instructions.

### FIELD OF THE INVENTION

The present invention relates to methods, equipment and data processing software for monitoring the body of a test subject during physical exercise. In a typical but non-limiting use case, the test subject is also the user of the equipment during exercise.

### BACKGROUND OF THE INVENTION

EP1795128 discloses methods, hardware and software for determining performance power of a physical exercise by means of registered user movement data, and for determining an exertion level corresponding to the performance power by means of physiological information measured from the user.

A limitation in the technique of EP1795128 is that the performance power is derived from velocity by using a generic conversion factor which may not be accurate for an individual user. For instance, the technique of EP1795128 permits determining parameters like velocity, performance power, exertion level, or the like, of multiple users. But the technique of EP1795128 is poorly equipped to indicate the reasons why some users are less efficient than others.

### SUMMARY OF THE INVENTION

It is an object of the present invention to alleviate one or more of the problems identified above. Specifically, it is an object of the present invention to provide methods, equipment and computer program products that provide improvements with regard to monitoring the body of a test subject during physical exercise. It is another object of the invention to help the test subject exercise in a manner which provides improvements over known techniques with respect to efficiency, elimination of injuries and/or elimination of excessive wear or fatigue.

The object of the invention is achieved with methods, data processing systems and software for data processing systems as defined in the attached independent claims. The following detailed description and the attached drawings present specific embodiments and implementations, some of which are presented in the attached dependent claims.

An aspect of the present invention is a method comprising performing a plurality of acts on a portable data processing system, wherein the portable processing system comprises:
a memory system for storing program code instructions and data, and a processing system for processing the data by executing the stored program code instructions;
a reception system for receiving data from a set of acceleration sensors, wherein the set of acceleration sensors is configured to indicate acceleration of the test subject in three orthogonal directions;
wherein the stored data comprises a plurality of conversion algorithms for converting measured parameters to processed parameters;
   wherein the plurality of acts performed on the data processing system comprises:
receiving acceleration sensor data from the set of acceleration sensors;
determining instantaneous vertical acceleration, instantaneous longitudinal acceleration and instantaneous lateral acceleration from the received acceleration sensor data;
identifying periodicity in one of the instantaneous accelerations and using the identified periodicity with the instantaneous accelerations, to determine one or more dynamic movement parameters related to movement of the test subject's body over a surface supporting the test subject's body;
wherein one or more of the dynamic movement parameters comprise a first energy-related value indicative of vertical energy loss, a second energy-related value indicative of brake energy loss, which are determined separately for at least two legs of the test subject and for a combination of the legs;
using the first energy-related value and second energy-related value to determine a third energy-related value indicative of combined energy loss; and
outputting an indication of the combined energy loss and/or correction instructions.

For the sake of brevity, the data processing system and the set of sensors are collectively called a performance monitor. Other aspects of the invention include the performance monitor or substantial subsets of it. An example of a whole performance monitor is a compact, preferably portable, data processing device with appropriate software, sensors and interconnections. An example of a substantial subset of the performance monitor is a software package with the set of sensors and interconnections for converting an existing data processing device to an embodiment of the inventive performance monitor. The data processing device is preferably a portable one, such as a smartphone, palm computer, tablet computer, smart watch or a dedicated pocket-sized data processing device. For the sake of completeness, it should be mentioned that portability is not a mandatory requirement for the data processing device. For instance, in some implementations, the data processing device may be integrated in a treadmill. In other implementations, a portable communication device may relay measurement data to, and optionally receive real-time feedback from an external data processing device.

In the context of the present invention, the test subject is a human or nonhuman animal the body of which is monitored during the physical exercise. In a typical use case, the test subject is also the user of the performance monitor, but the invention is not limited to such use cases, and the user can be a distinct person from the test subject.

### BRIEF DESCRIPTION OF THE DRAWINGS

In the following section, specific embodiments of the invention will be described in greater detail in connection with illustrative but non-restrictive examples. A reference is made to the following drawings:

FIG. 1 shows an exemplary data processing architecture suitable for implementing embodiments of the invention;

FIG. 2 shows a schematic block diagram of a mobile terminal, which can be used for data processing tasks in some embodiments of the invention;

FIGS. 3 and 4 illustrate how observations of the sensors are progressively processed into more valuable information in an embodiment specifically adapted for running;

FIG. 5 illustrates instantaneous vertical acceleration as a function of time and various ways to determine periodicity;

FIG. 6 illustrates instantaneous longitudinal acceleration in a common time reference with FIG. 6;

FIG. 7 illustrates determination of upper body inclination;

FIG. 8 illustrates determination of additional optional derivative quantities;

FIGS. 9, 10 and 11 are graphs that illustrate analysis of body dynamics; and

FIG. 12 illustrates calculation of actual and optimal ratios between longitudinal and vertical acceleration forces; and

FIG. 13 illustrates calculation of actual and optimal ratios between brake force and vertical force at the time of strike (impact) on the supporting surface.

### DETAILED DESCRIPTION OF SOME SPECIFIC EMBODIMENTS

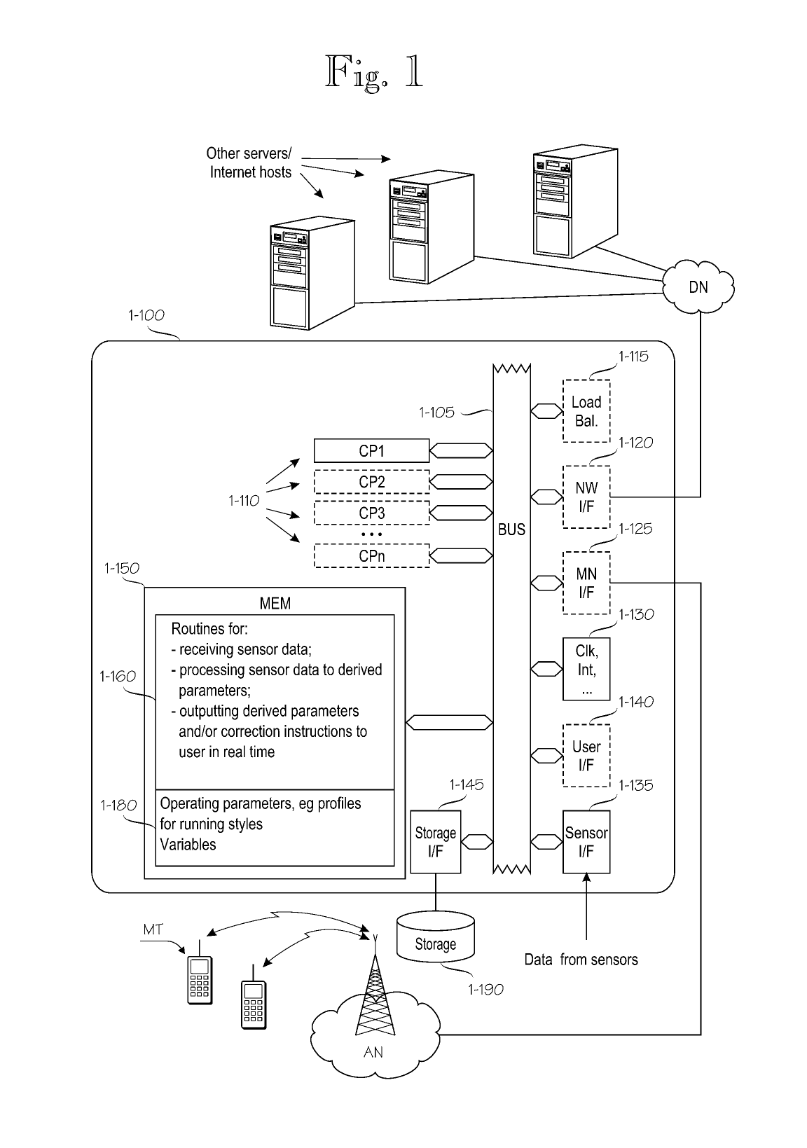

In the attached drawings, boxes shown with a dashed outline generally denote optional elements, which provide additional features and/or solve additional problems. FIG. 1 shows an exemplary data processing architecture that can be programmed to perform the various data processing tasks relating to embodiments of the invention. In the following the data processing architecture will be referred to as a computer, but those skilled in the art will realize that the data processing architecture need not be implemented as a dedicated computer. Instead, several embedded techniques are possible, as are techniques in which the inventive functionality is installed on a data processing system that exists for other purposes.

The architecture of the computer, generally denoted by reference numeral 1-100, comprises one or more central processing units CP1 . . . CPn, generally denoted by reference numeral 1-110. Embodiments comprising multiple processing units 1-110 are preferably provided with a load balancing unit 1-115 that balances processing load among the multiple processing units 1-110. The multiple processing units 1-110 may be implemented as separate processor components or as physical processor cores or virtual processors within a single component case. In a typical implementation the computer architecture 1-100 comprises a network interface 1-120 for communicating with various data networks, which are generally denoted by reference sign DN. The data networks DN may include local-area networks, such as an Ethernet network, and/or wide-area networks, such as the internet. In some implementations the computer architecture may comprise a wireless network interface, generally denoted by reference numeral 1-125. By means of the wireless network interface, the computer 1-100 may communicate with various access networks AN, such as cellular networks or Wireless Local-Area Networks (WLAN). Other forms of wireless communications include short-range wireless techniques, such as Bluetooth and various “Bee” interfaces, such as XBee, ZigBee or one of their proprietary implementations.

The computer architecture 1-100 may optionally comprise a local user interface 1-140. Depending on implementation, the user interface 1-140 may comprise local input-output circuitry for a local user interface, such as a keyboard, mouse and display (not shown). A wearable display, such as eyeglasses with training data superimposed on the user's field of vision, can be advantageous in connection with embodiments of the the present invention.

The computer architecture also comprises memory 1-150 for storing program instructions, operating parameters and variables. Reference numeral 1-160 denotes a program suite for the server computer 1-100.

The computer architecture 1-100 also comprises circuitry for various clocks, interrupts and the like, and these are generally depicted by reference numeral 1-130.

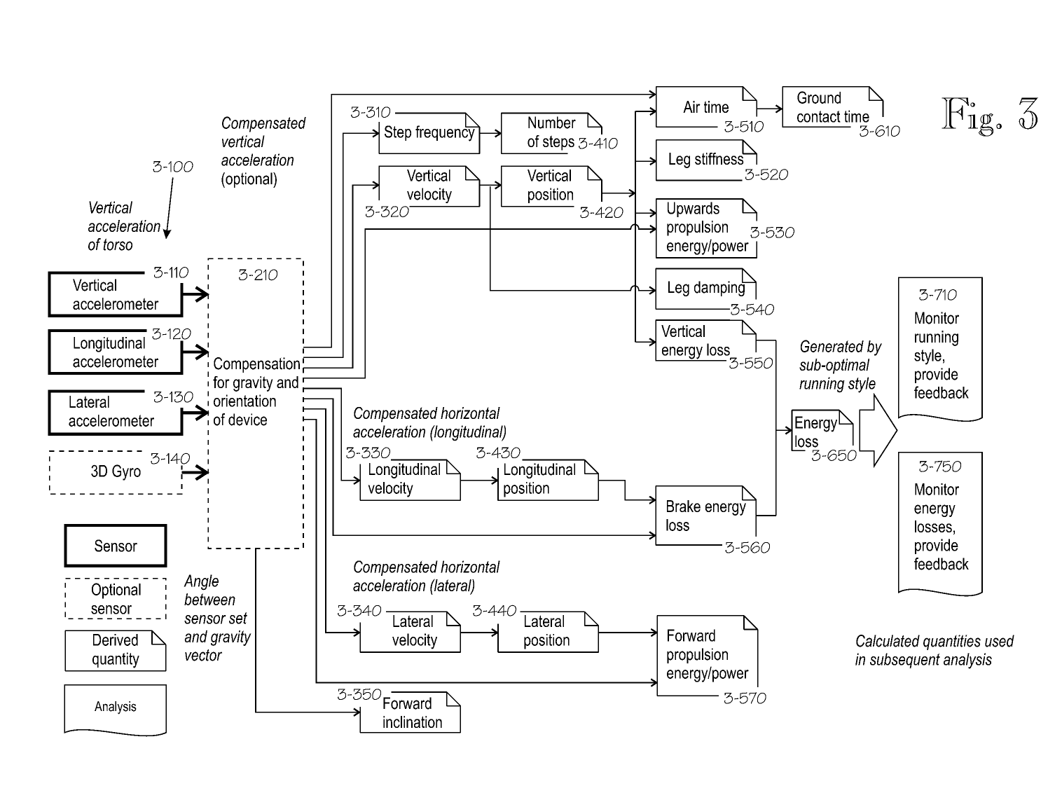

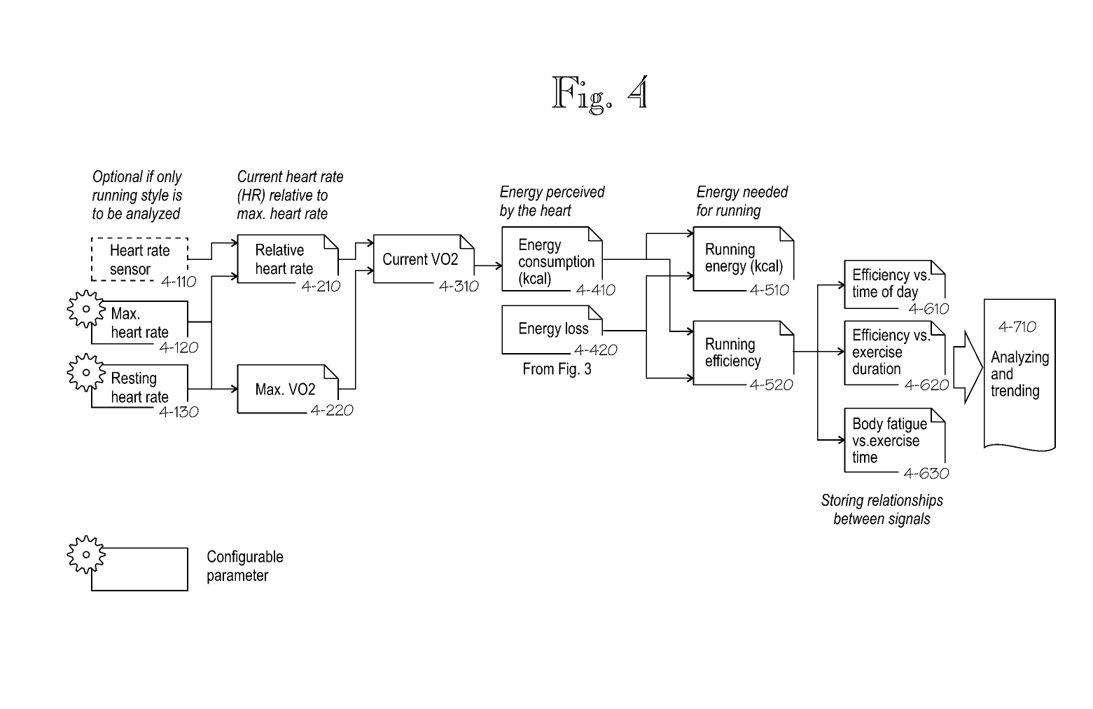

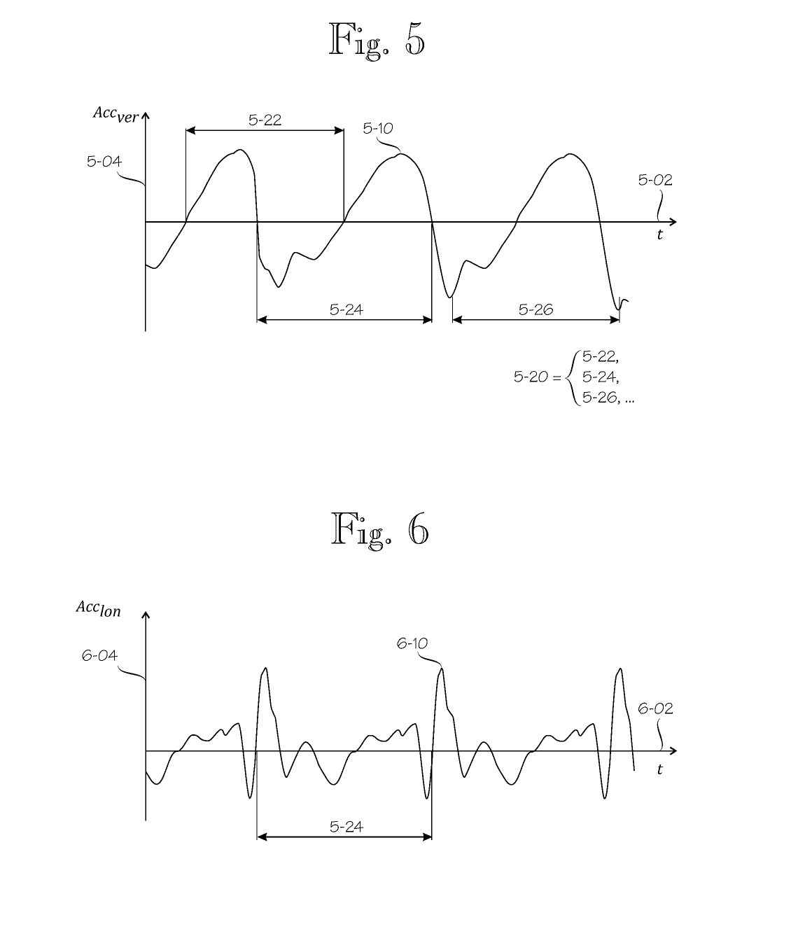

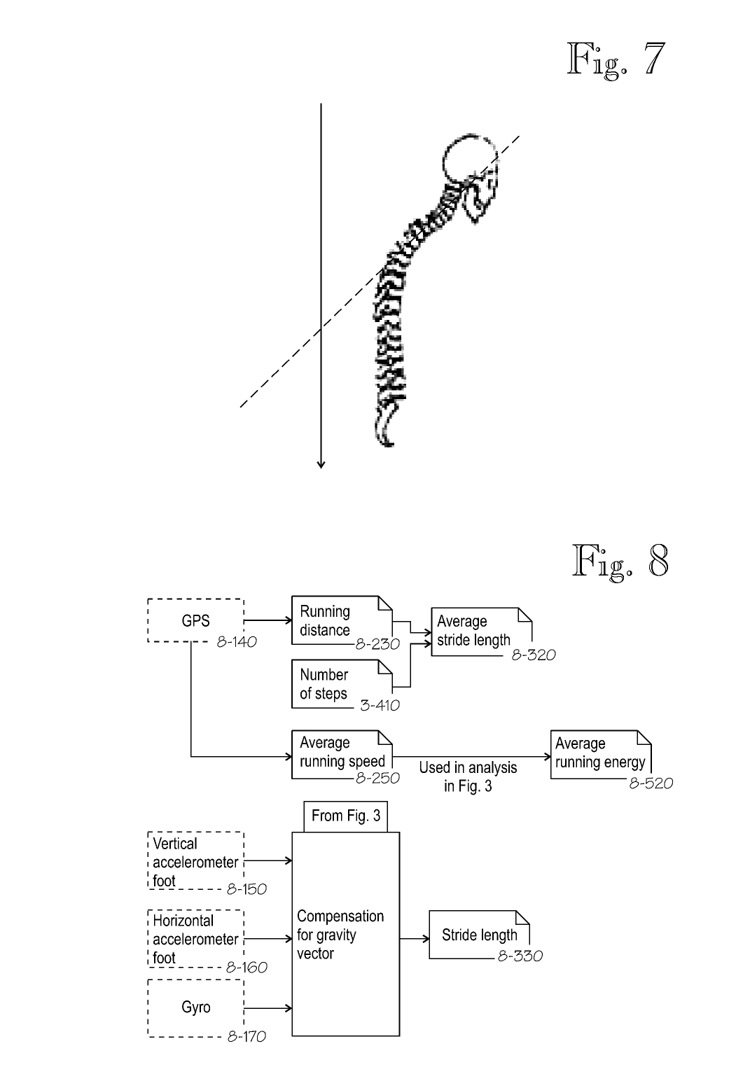

Reference number 1-135 denotes an interface by which the computer obtains data from various sensors, such as the heartbeat sensor, acceleration sensors and so on. The interface 1-135 is preferably but not necessarily a wireless interface whereby it restricts movement of the test subject as little as possible. Typical but non-restrictive forms of wireless communications include short-range wireless techniques, such as Bluetooth and various “Bee” interfaces, such as XBee, ZigBee or one of their proprietary implementations. The set of sensors will be further described in connection with FIGS. 3 and 4.

The computer architecture 1-100 further comprises a storage interface 1-145 to a storage system 1-190. The storage system 1-190 comprises non-volatile storage, such as a magnetically, optically or magneto-optically rewritable disk and/or non-volatile semiconductor memory, commonly referred to as Solid State Drive (SSD) or Flash memory. When the computer is switched off, the storage system 1-190 may store the software that implements the processing functions, and on power-up, the software is read into semiconductor memory 1-150. The storage system 1-190 also retains operating data and variables over power-off periods. The various elements 1-110 through 1-150 intercommunicate via a bus 1-105, which carries address signals, data signals and control signals, as is well known to those skilled in the art.

The inventive techniques may be implemented in the computer architecture 1-100 as follows. The program suite 1-160 comprises program routines or program code instructions for instructing the processor or set of processors 1-110 to execute the functions of the inventive method or its embodiments, including: receiving sensor data; processing sensor data to derived parameters and outputting derived parameters and/or correction instructions to the user (test subject) in real time.

In addition to instructions for carrying out a method according to the invention or its embodiments, the memory 1-160 stores instructions for carrying out normal system or operating system functions, such as resource allocation, inter-process communication, or the like.

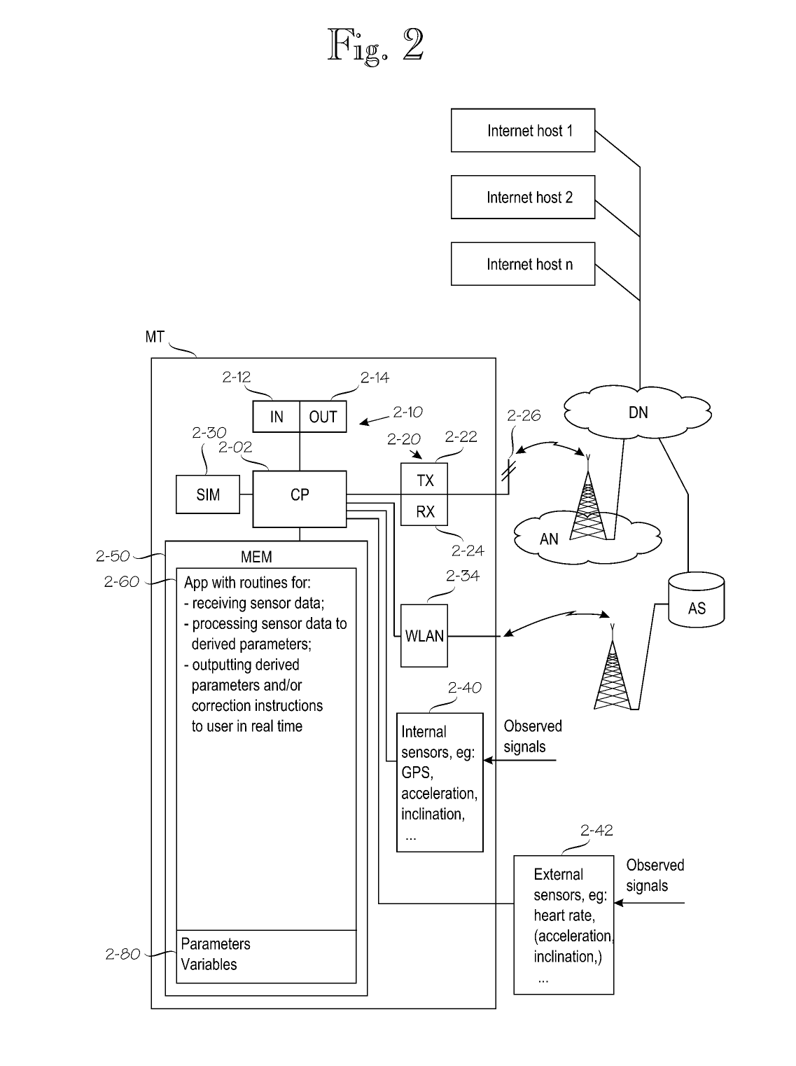

FIG. 2 shows a schematic block diagram of a mobile terminal, which can be used for data processing tasks in some embodiments of the invention. The mobile terminal MT comprises a processing system 2-02 with at least one central processing unit. The mobile terminal further comprises a memory system 2-50, which typically comprises a combination of fast volatile memory and slower non-volatile memory, as is well known to those skilled in the art. In addition, the mobile terminal MT comprises or utilizes a user interface 2-10, which comprises an input circuitry 2-12 and an output circuitry 2-14. The input circuitry 2-12 comprises the mobile terminal's microphone and user-input devices, such as a keypad and/or touch screen. The output circuitry 2-14 comprises the mobile terminal's display and earphone or loudspeaker. The mobile terminal MT further comprises reception/transmission circuitry 2-20 which comprises a transmission circuitry 2-22, reception circuitry 2-24 and antenna 2-26. A subscriber identity module, SIM, 2-30 is used by an authentication function to authenticate the mobile terminal's user and to identify the user's subscription to the access network AN. A typical modern mobile terminal also comprises WLAN (Wireless Local Area Network) circuitry 2-34 whose normal mode of usage is acting as a WLAN client to a WLAN base station (not shown).

In order to support installable program modules, the mobile terminal's memory 2-50 typically comprises routines for downloading installable program modules and for storing the installable program modules as apps (applications) 2-60 in the memory 2-50 for execution by the central processing unit CP. FIG. 2 shows an arrangement in which the mobile terminal is configured to download installable program modules from an app store ASn (n=1, 2-, 3, . . . , depending on the platform) via a data network DN, an access network AN, the antenna 2-26 and reception circuitry 2-24. Instead of downloading software from the app store over the access network, or in addition to it, other arrangements are equally possible, such as downloading the installable program modules via the data network DN to a separate data terminal (not shown), from which the installable program modules are transferred to the mobile terminal the WLAN circuitry 2-34 or via some other short-range connection, such as Bluetooth or Universal Serial Bus (USB, not shown separately). The access network AN is typically a broadband-capable mobile communication network, while the data network DN is typically the internet or some closed subnetwork implementing internet protocol (IP), commonly called intranets or extranets. At this level of generalization, all previously-discussed elements of FIG. 2 can be conventional as used in the relevant art. One or more external hosts are accessible via the access network AN and data network DN, as will be described in more detail below. Finally, reference numeral 2-80 denotes an area of the memory 2-50 used to store parameters and temporary variables.

In addition to the user interface 2-10, the mobile terminal typically comprises a set of internal sensors 2-40 for detecting environmental variables or parameters. A non-exhaustive list of sensors 2-40 includes a camera, IR (infrared) detection/communication circuitry, GPS and/or other location-determination circuitry, compass, gyroscope (inclination sensor), RFID (radio frequency identification) and/or NFC (near-field communication) circuitry, or the like. In the present context, internal sensors refer to sensors built in to the mobile terminal. For instance, the GPS sensor can indicate the runner's position, velocity, orientation and altitude. The orientation can alternatively or additionally obtained from the compass, if available. The inclination sensor, if available, can be used to measure how much the runner's upper body deviates from true vertical direction, provided that the mobile terminal is mounted in a known inclination with regard to the upper body. The idea is to determine the inclination of the upper spine, but for obvious reasons the mobile terminal cannot be attached to the spine. Assuming that the runner's chest deviates from the inclination of the spine by nn degrees, those nn degrees can be taken into account when determining the inclination of the spine.

Information on altitude changes from the GPS sensor can be used to correct calculations which are accurate for level ground. For instance, uphill running consumes more energy that running on level ground.

The set of internal sensors 2-40 of a typical mobile terminal is usually insufficient for the purposes of the present invention and/or its embodiments. Accordingly the set of internal sensors is complemented by a set of external sensors, collectively referred to by reference number 2-42. In an illustrative but nonrestrictive implementation, the set of external sensors 2-42 comprises a heart rate monitor (sensor), a multi-dimensional acceleration sensor for each foot, inclination sensors in addition to the one that may be pre-installed in the mobile terminal, or the like.

FIG. 3 illustrates how observations of the sensors are progressively processed into more valuable information in an embodiment specifically adapted for running. Other types of sports may require modifications to the nature, number and/or placement of sensors, and to the algorithms by which derived quantities are obtained from the output of the sensors.

In the embodiment shown in FIG. 3, the set of sensors is collectively denoted by reference number 3-100. Depending on implementation, some sensors are mandatory and some are optional. In an embodiment specifically adapted for running, the set of mandatory sensors include a set of accelerometers mounted on the test subject's torso or limbs during use. In a typical but non-restrictive implementation, this set of sensors comprise three accelerometers 3-110, 3-120, 3-130, respectively configured to measure vertical acceleration, forward acceleration and lateral acceleration. Vertical means up-down, forward means in the direction of running, and lateral means sideways (left-right). Instead of three unidirectional accelerometers, acceleration along three axes can be measured by one three-dimensional (3D) accelerometer, for example. A 3D gyro (orientation/inclination) sensor 3-140 is an example of an optional sensor. The term “gyro” is originally derived from an electro-mechanical gyroscope, but the term is now being used colloquially to refer to solid-stated orientation/inclination sensors.

Reference number 3-210 denotes an optional gravity/orientation compensation block. The gravity/orientation compensation block 3-210 is configured to compensate for gravity and orientation error(s) of the sensors. In the present context, orientation errors refer to the angle between true vertical, forward and sideways axes and the corresponding orientations of the respective sensor axes.

Reference numbers 3-310 through 3-350 denote a set of quantities derived from the sensor outputs by the optional gravity/orientation compensation block 3-210. In the present embodiment, this set of derived quantities comprises step frequency (cadence) 3-310, vertical velocity 3-320, longitudinal (forward) velocity 3-330, lateral (sideways) velocity 3-340, and forward inclination 3-350, plus any quantities derived therefrom.

In the present implementation, the step frequency 3-310 (steps per unit time) and time since activation of the apparatus/application is used to derive the number of steps 3-410 since activation. In some publications, step frequency is called “cadence”.

The vertical velocity 3-320 can be derived by integration from the output of the accelerometer in the gravity/orientation compensation block 3-210. In an idealistic case wherein the three axes of the accelerometers 3-110, 3-120, 3-130 perfectly coincide with true vertical, forward and sideways directions, the vertical acceleration can be obtained from the vertical accelerometer 3-110 alone. But in the real world, the axes of the accelerometers deviate from the ideal cases, and the vertical acceleration can be obtained from the outputs of the acceleration sensors 3-110, 3-120, 3-130 and the 3D gyro 3-140. Vertical acceleration in the upwards direction (opposite to the direction of gravity) can be computed by simple trigonometry from the outputs of the acceleration sensors and their orientations. Obviously the orientation of the 3D gyro 3-140 must coincide with the three acceleration sensors 3-110, 3-120, 3-130. This is always the case if the 3D gyro 3-140 and the three acceleration sensors 3-110, 3-120, 3-130 are integrated into a single physical device.

In embodiments of the invention specifically adapted for running, vertical acceleration is a cyclic quantity that determines the periodicity for other acceleration-based calculations and analyses. This periodicity will be further illustrated in connection with FIG. 5.

FIG. 5 illustrates instantaneous vertical acceleration as a function of time. The horizontal axis 5-02 corresponds to time, while the vertical axis 5-04 corresponds to instantaneous vertical acceleration, with optional compensation for the orientation of the vertical accelerometer(s), as described earlier. Graph 5-10 schematically depicts instantaneous vertical acceleration.

In addition to serving as a useful input quantity for several derived quantities, vertical acceleration indicates the phase of the step that the runner is currently in. In sports whose performance essentially consists of repetitions of similar movements, accelerations exhibit clear periodicity. Accordingly, the graph 5-10 is cyclical. The inventors have discovered that in running, the clearest periodicity can be detected in the vertical acceleration. The period of vertical acceleration can be defined between any two recurring points or shapes in the vertical acceleration 5-10. By way of example, FIG. 5 shows three ways to define and detect a period. Reference numbers 5-22 and 5-24 show periods that respectively occur between two consecutive zero crossings of the upward or downward slope of the vertical acceleration 5-10. Reference number 5-26 shows a period that occurs between two consecutive negative peaks of the vertical acceleration 5-10. In theory, a negative peak can be detected as the point in time when the first derivative of vertical acceleration changes from negative to positive. In practice it is better to define the negative peak as a point in time when the first derivative (slope) of vertical acceleration changes from negative to positive, and reaches a finite threshold value. As can be seen from FIG. 5, the timing of the positive peaks is not as clearly defined as the negative peaks or the zero crossings and, accordingly, are not equally well suited to act as timing references.

Returning to FIG. 3, cadence or step frequency is calculated by block 3-310. The step frequency is defined for each and every step by measuring the acceleration of the body in vertical direction, as was described in connection with FIG. 5. While the calculations required to determine the step period (inverse of step frequency) are basically simple, an additional challenge is posed by the fact that several platforms provided by existing data processing devices, such as smartphones, do not have an application programming interface (“API”) that offers a universally available timer with better than one-second resolution. In the present context “universally available” means that the same timer-related code could be used across the entire platform of brand X. In practice, however, many platforms offer “TickCount” (Symbian term) or similar functions but the relation between “TickCount” and seconds depends on the model of the device being used. Fortunately, smartphones can convert the instantaneous vertical acceleration to speech encoding by well-standardized encoding schemes. The speech encoding yields the number of samples in each period, whereby the length of the period is (number of samples)*(sample frequency). The step frequency is then 1/period.

Vertical velocity is calculated by block 3-320. The vertical velocity can be calculated from vertical acceleration by integration, which in a digital implementations is often carried out by summing, using discrete periods:

Vᵥₑᵣ[k]=Vᵥₑᵣ[k−1]+Accᵥₑᵣ[k]*sample period

Herein Vᵥₑᵣ[i] and Accᵥₑᵣ[i] are vertical velocity and acceleration at sample i, while sample period is the duration of the discrete periods.

Vertical position is calculated by block 3-420. Vertical position can be calculated from vertical velocity by integration, which in a digital implementations is often carried out by summing, using discrete periods:

POSᵥₑᵣ[k]=POSᵥₑᵣ[k−1]+Vᵥₑᵣ[k]*sample period

Herein POSᵥₑᵣ[i] and Vᵥₑᵣ[i] are vertical position and velocity at sample i, while sample period is the duration of the discrete periods. The vertical movement ΔPOSᵥₑᵣ is then given by:

ΔPOSᵥₑᵣ=POSᵥₑᵣ_{_{—}}ₘₐₓ−POSᵥₑᵣ_{_{—}}ₘᵢₙ

Herein POSᵥₑᵣ_{_{—}}ₘₐₓ−POSᵥₑᵣ_{_{—}}ₘᵢₙ are the maximum and minimum values for vertical position over a period.

Air time and ground contact time are calculated by blocks 3-510 and 3-610, respectively. Air time is defined as the time when the runner is not supported by the ground underneath. Without support from below, vertical acceleration ACCᵥₑᵣ is −g. In other words, the difference between measured acceleration Accᵥₑᵣ and −g is zero. Ground contact time is time which is not air time.

As regards vertical acceleration, one should keep in mind that when not supported by the ground, the runner—or the runner's center of gravity—is in free fall, with Accᵥₑᵣ=−g, as seen by an external observer. Yet an acceleration sensor carried along with the runner registers Accᵥₑᵣ=0 because the enclosure of the acceleration sensor does not experience any external forces acting on it. In contrast, when a stationary acceleration sensor is in a gravity field, it registers an acceleration of −g.

Leg-wise stiffness and its leg-wise ratio are calculated in block 3-520. In a somewhat simplistic implementation, the vertical force Fᵥₑᵣ at each sample may be given by:

Fᵥₑᵣ=ACCᵥₑᵣ·m

Herein m is the body mass. By summing all the Fᵥₑᵣ with a positive sign for each leg separately, the left-to-right ratio between the vertical propulsion work produced by the two legs can be calculated as:

Fᵥₑᵣ_{_{—}}ᵣₐₜᵢₒ=Fᵥₑᵣ_{_{—}}_{left}/Fᵥₑᵣ_{_{—}}_{right}

In an analogous manner, the ratio between the braking work in vertical direction can be defined by summing the negative forces and dividing the leg-wise values. The following holds for the slightly simplified case wherein the runner's body is modeled as a single mass in the vertical direction. The stiffness of the legs K can be calculated as:

K=Fᵥₑᵣ_{_{—}}ₘₐₓ/POSᵥₑᵣ_{_{—}}ₘₐₓ

Herein Fᵥₑᵣ_{_{—}}ₘₐₓ is the maximum value of Fᵥₑᵣ during a cycle, while POSᵥₑᵣ_{_{—}}ₘₐₓ is the maximum negative position during the cycle. As was described in connection with FIG. 5, the runner's body exhibits a cyclic vertically oscillating behavior. It is possible to normalize the quantities by using average values of the quantities during the cycle as “zero” (reference values for normalization). By means of discrete integration it is possible to calculate the vertical velocity and position. In this context “maximum negative position” is the lowest value of the vertical position during the cycle.

Vertical (upwards) propulsion work is calculated by block 3-530 for each leg separately. Based on the vertical acceleration Accᵥₑᵣ, the ratio or balance between the positive vertical propulsion force can be calculated. In a typical but non-restrictive implementation, the accelerations are measured on the torso (upper body). When the test subject is running, at most one leg at a time contributes to the runner's vertical and horizontal accelerations. This means that the accelerations of the torso are alternatively driven by the left and right leg. The apparatus of the invention does not necessarily know whether the first observed step was taken by left or right leg, but it doesn't matter. It is nevertheless possible to calculate the various leg-wise ratios, such as the ratio of the leg-wise stiffness.

Leg damping and its left-to-right ratio are calculated in block 3-540. Leg damping can be calculated in a manner analogous with leg stiffness, by replacing vertical position of the body by the vertical speed of the body. Moreover, the left-to-right ratio of these leg-wise parameters can be determined by dividing the quantities of the left leg with those of the right leg.

Vertical energy loss is calculated by block 3-550. Potential energy Eₚₒₜ used during a period is calculated as:

Eₚₒₜ=m·g·ΔPOSᵥₑᵣ

Herein m is the body mass and g is the gravity constant (typically 9.81 m/s²). Since the change in potential energy in vertical direction does not contribute to forward motion of the runner, it is treated as a pure energy loss in a simple implementation. More ambitious implementations may take elasticity of the human body into account and reduce energy loss thus calculated accordingly.

FIG. 6 illustrates instantaneous longitudinal acceleration as a function of time. In the present context, longitudinal means the forward direction of principal motion. For instance, in the case of running, longitudinal acceleration, motion and position refer to the direction of running. In FIG. 6, the horizontal axis 6-02 corresponds to time, while the vertical axis 6-04 corresponds to instantaneous longitudinal acceleration, with optional compensation for the orientation of the vertical accelerometer(s), as described earlier. Graph 6-10 schematically depicts instantaneous vertical acceleration. FIGS. 5 and 6 use the same time scale and the periodicity is the same, at least over a sufficiently long observation period. By way of example, period 5-24 has been copied from FIG. 5 to FIG. 6. Although, in connection with FIG. 5, period 5-24 was defined as the period between two consecutive downwards zero crossings, it almost coincides with two consecutive upwards zero crossings in FIG. 6.

Returning again to FIG. 3, longitudinal horizontal acceleration is obtained from longitudinal horizontal acceleration sensor 3-130 whose output is optionally corrected for the discrepancy between the orientation of the sensor and the direction of gravity. This takes place in compensation block 3-210. From the corrected longitudinal acceleration, longitudinal velocity is calculated by integration or discrete summing in block 3-330. Longitudinal position is calculated from longitudinal velocity in block 3-430. Mathematically, calculations of longitudinal velocity and position are analogous with calculations of vertical velocity and position, which were described earlier, in connection with respective blocks 3-320 and 3-340. In short, velocity can be derived from acceleration by integration or discrete summing, and position can be derived from velocity by integration or discrete summing.

Similarly, lateral (sideways) acceleration is obtained from lateral acceleration sensor 3-120 whose output is optionally corrected for orientation in compensation block 3-210. Lateral velocity and position are calculated in respective blocks 3-340 and 3-440 by integration or discrete summing, as was described in connection with longitudinal and vertical acceleration, velocity and position.

FIG. 7 shows a runner's upper body (skull and spine) with a slight forward lean with reference to the direction of the gravity vector. By installing a set of accelerometers on the upper body of the test subject, the posture of the upper body can be determined by comparing the orientation of the set of accelerometers with the direction of the gravity vector.

Returning again to FIG. 3, block 3-350 determines forward inclination of a runner's upper body. This information can be used to to produce sensory feedback to the test subject during exercise. By way of example the sensory feedback can comprise any combination of visible, audible and tactile information. The visible, audible and/or tactile information provided to the test subject helps the test subject correct their posture during exercise, and thus reduces the risk of excessive fatigue or wear or injury.

Reactive brake work (brake energy loss) and power are calculated in block 3-560. When the test subject's foot strikes the ground, reactive forces are generated in the vertical direction and the direction opposite to forward motion. Especially the brake force opposite to the direction of the forward motion results in energy losses. The reactive brake force is determined for the segment indicated in FIG. 6. The first negative point indicates where the foot first strikes the ground and is usually the most accentuated. In a somewhat simplistic model, wherein the runner's body is modeled by a single mass, the brake force F_{brake} can be calculated from longitudinal acceleration Accᵢₒₙ and body mass m by multiplication:

F_{brake}=ACCᵢₒₙ·m

The reactive brake work is then calculated as a product of force and distance. In one illustrative example, for each sample, such as the time segment 5-24 shown in FIGS. 5 and 6, the force is multiplied with the distance traveled since the previous sample, and the products are summed for the relevant time period. The reactive brake power can now be calculated as the reactive brake work divided by the time during which the work was produced. Alternatively or additionally, the smallest value of the horizontal acceleration could be used for indicating the level of energy loss during the foot strike. The positive work from each leg can be calculated a similar manner.

Longitudinal propulsion work and its leg-wise ratio are calculated in block 3-570. These calculations can be carried out similarly to the calculations performed by block 3-560.

Similar to the vertical propulsion work ratio, the ratio between the work done in horizontal direction of the movement can be calculated.

Block 3-650 calculates total energy loss of the test subject as the sum of the vertical energy loss (from block 3-550) and brake energy loss (from block 3-560) for each leg separately. The sum of both legs then represents the total energy loss for the whole step cycle. The level of energy loss can then be analyzed with respect to a pre-defined level or based to a user-defined level.

Reference numbers 3-710 and 3-750 denote two exemplary analysis sections that provide the test subject with helpful information, preferably during exercise. By way of example, reference number 3-710 denotes an exemplary analysis section, which is configured to identify the test subject's current running style, or accept a selection of running style from the user interface, and then helps the test subject to maintain the identified or selected running style. Reference number 3-750 denotes an exemplary instruction block that is configured to provide the test subject with real-time instructions that help the test subject to minimize energy losses and/or correct body position. Such real-time instructions help minimize energy losses, risk to injury, excessive fatigue or wear, and so on.

FIG. 4 illustrates various optional sensors and processing blocks for calculating various parameters that relate to the test subject's metabolic efficiency. The primary inputs in the block diagram of FIG. 4 are the test subject's heart rate HR, as indicated by heart rate sensor 4-110, plus a number of configurable parameters, such as maximum heart rate HR. 4-120 and resting heart rate HR_{Rest} 4-130. The configurable parameters can be inputted and adjusted via the user interface of the computer or data processing system (cf. items 1-140, 2-10 in FIGS. 1 and 2). Instead of inputting maximum heart rate directly, it is possible to estimate the maximum heart rate HRₘₐₓ from the test subject's age in years, Num_{years}, by an experimental formula, such as:

HRₘₐₓ=208−0.7·Num_{years}

Herein, 208 and 0.7 are approximate values which are valid for healthy persons. Instead of inputting resting heart rate directly, it can be measured by the heart rate sensor 4-110 at a time when the test subject is at rest.

From the primary inputs, a relative heart rate is calculated in block 4-210 as a ratio of current heart rate to maximum heart rate.

Block 4-220 calculates the maximum amount of oxygen transported with the blood from another experimentally-obtained formula:

V_{O2}_{_{—}}ₘₐₓ=15.3·(HRₘₐₓ/HR_{Rest})(ml/kg/min)

Block 4-310 calculates the test subject's current V_{O2} level from yet another experimentally-obtained formula:

V_{O2}=(((HR/HRₘₐₓ)−37.182)/0.6463)·V_{O2}_{_{—}}ₘₐₓ

From the current V_{O2} level the load experienced by the body P_{body} is calculated in block 4-410. In an illustrative but non-limiting example, the calculation uses the following experimentally-obtained formula:

P_{body}=1.5·V_{O2}=4.924·T_{exercise}.

Herein T_{exercise} is the duration of the present exercise in minutes. Block 4-520 is now able to calculate the efficiency Eff by which the load experienced by the body P_{body} is converted to forward motion, as a function of the power losses Pₗₒₛₛ as:

Eff=(P_{body}−Pₗₒₛₛ)/P_{body}.

Alternatively, P_{body} can also be determined as a linear function of the general running speed. This is of course less accurate than the method described above.

Now that the efficiency of the body and the running style have been calculated for each step, the data processing system is able to map the efficiency against various quantities. For example, optional block 4-610 calculates efficiency as a function of time of the day. Another optional block 4-620 calculates efficiency as a function of the duration of the exercise, and a third optional block 4-630 calculates body fatigue versus duration of the exercise. In an analogous manner, it is possible to calculate yet further derived quantities, such as efficiency as a function of date, which indicates development of efficiency as the test subject's fitness improves over the course of exercises. This also applies to other use cases in which the heart rate and the actual work are measured or estimated, such as in fitness equipments.

FIG. 8 illustrates determination of additional optional derivative quantities. A GPS device 8-140, if present, can be used to indicate a current value for running distance 8-240. Block 3-410, which was described in connection with FIG. 3, outputs the number of steps. Block 8-320 determines average stride length by dividing the running distance 8-240 by the number of steps 3-410. The output of the GPS device 8-140 indicates, or it can be used to derive, average running speed 8-250, which can be used to determine various energy-related parameters, which are collectively denoted by reference number 8-250 and which were described in detail in connection with FIG. 3.

Alternatively or additionally, an instantaneous value for stride length 8-330 can be derived from gravity- and orientation-compensated outputs from vertical and horizontal acceleration sensors 8-150, 8-160 mounted on the test subject's foot or feet. Compensation for gravity and orientations uses output from gyro (inclination sensor) 8-170.

FIGS. 9, 10 and 11 are graphs that illustrate dynamic monitoring of metabolic process of the test subject. Referring to FIG. 9, when an exercise is initiated the actual VO2 level increases more slowly than what is actually needed for the exercise. This difference, called oxygen deficit, is denoted by reference number 9-10. FIG. 10 shows a comparable graph for high-intensity training. The actual exercise demands more oxygen than can actually be provided, and oxygen deficit, now denoted by reference number 10-10, is much bigger than in FIG. 9. Various interval-training schemes have been developed in order to improve the ability of the body to quickly maintain high levels of VO2. In general, the better trained a person is, the faster the VO2 level rise and the lower the oxygen deficit will be. There are of course a number of other factors affecting on this dynamics, such as nutrition.

For professional athletes, it is beneficial to understand the dynamics of their metabolism, and to be able to monitor it, during training programs. The following parameters can now be calculated:
Theoretical VO2 (VO2 level actually required to sustain the current exercise level)
Oxygen deficit
EPOC (Excess post exercise oxygen consumption)
Gain and Time constant of the VO2 process
Dead time (response time), ie, time delay from a change in stimulus to response, such as time from a load increase to a detected change in heart rate.

Using a mobile platform, targets for these parameters can be set and monitored during exercise. The test subject can be provided with information on deviations from the targets. This helps optimize the entire training program. This type of support can be beneficial for training athletes in sports which exhibit sudden high intensity movements followed by rest periods, such as football or ice-hockey.

Moreover, use of this information during games will enable coaches to optimize the schedule by which exhausted players will be replaced by rested ones. Comparison of the current time constant with the average time constant from previous exercises can also be used as an indication that the body is not well rested.

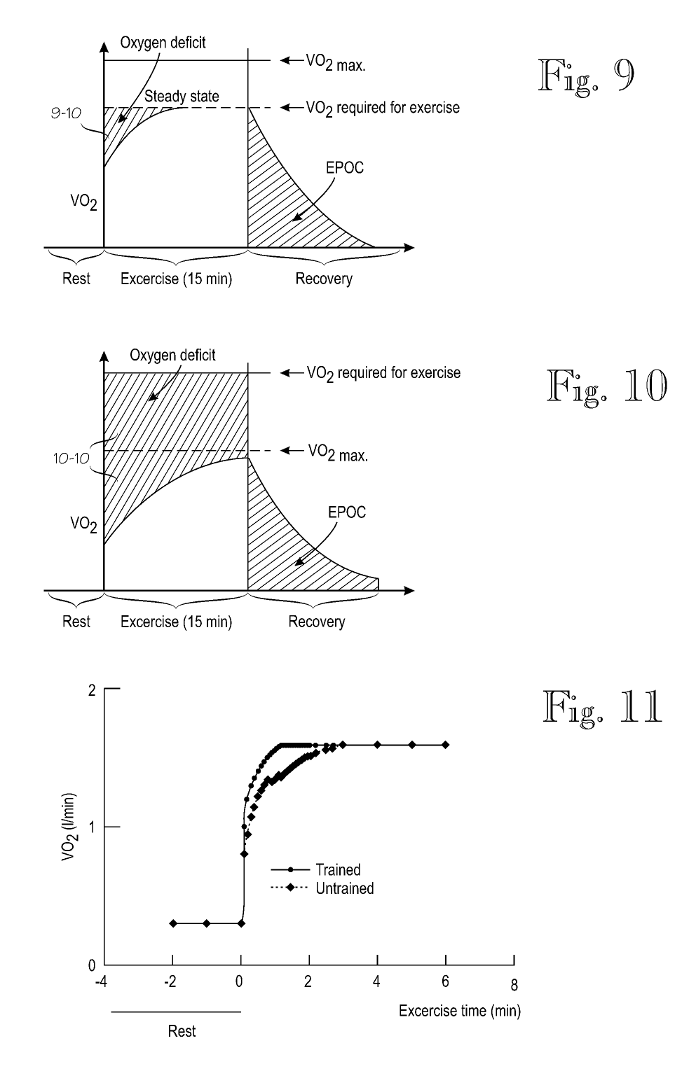

FIG. 11 shows a comparison of oxygen intake capability, V_{O2}, between a trained person and an untrained one. As previously presented, the average speed of the runner and a theoretical V_{O2} value can be calculated by using a GPS or other measurements. For a more detailed description of energy needed for running, the reader is referred to EP1795128, for example.

For light exercises, the theoretical V_{O2} and the V_{O2} measured from heart rate should coincide. However, for sudden changes in exercise intensity or for high intensity running, in which anaerobic processes are involved, the theoretical V_{O2} exceeds the V_{O2} measured from heart rate. The oxygen deficit can now be calculated as a time integral of the difference between the theoretical and measured V_{O2}.

In some embodiments this calculation is triggered by a detection of a positive peak in the difference between the theoretical and measured V_{O2}.

As regards excess post exercise oxygen consumption, EPOC, it too can be calculated as the time integral of the difference between the theoretical and measured V_{O2}. In some embodiments this calculation is triggered by a detection of a negative peak in the difference between the theoretical and measured V_{O2}.

As regards gain and time constant of the V_{O2} process, assuming that the V_{O2} process is a first-order process, it can be described by two parameters: a gain value K and a time constant T. There are many ways to identify these parameters, but a simple example will be given in the following.

Suppose that the test subject initiates an exercise by rapidly accelerating to a steady pace. When the positive peak in the difference between the theoretic and measured V_{O2} is registered, the measured V_{O2} levels are kept in memory until a steady state has been achieved. The time constant is now calculated as the time between the initiation of the exercise and to the point where the V_{O2} level has reached 1−1/e≈63.2% of the steady-state value. The process gain K is calculated as the measured steady-state V_{O2} level divided by the theoretical V_{O2}, cf. FIG. 11.

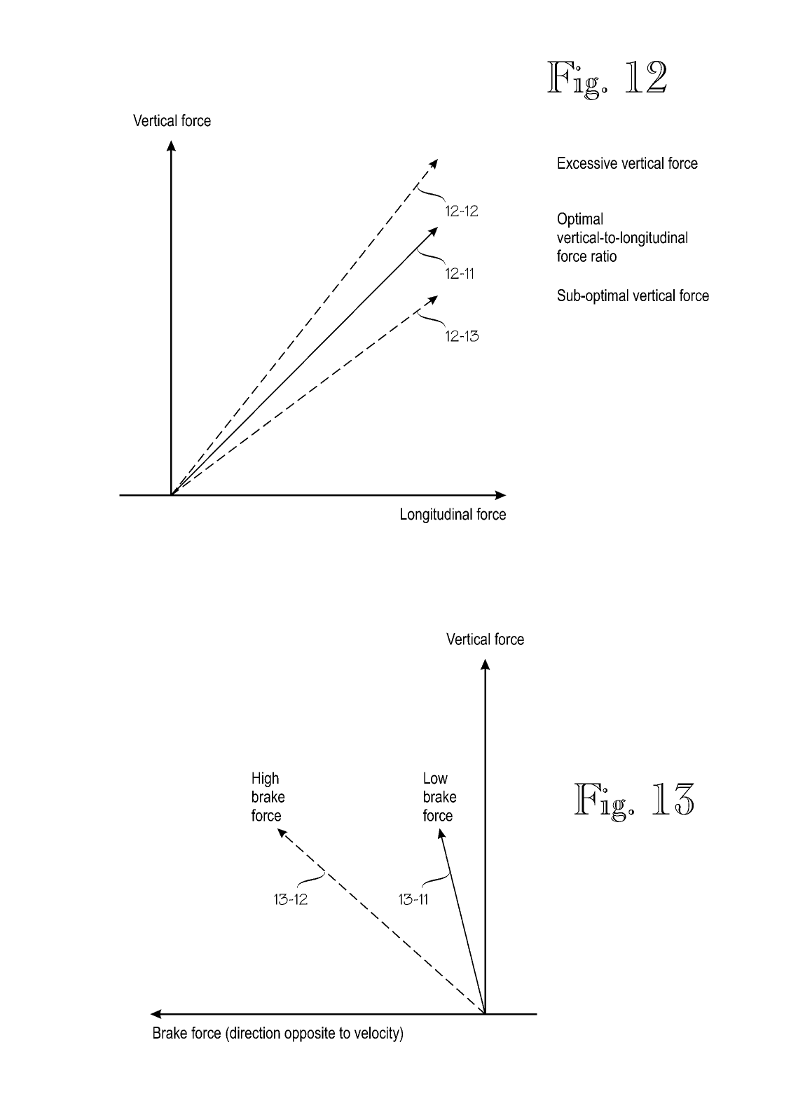

FIG. 12 illustrates calculation of actual and optimal ratios between longitudinal and vertical acceleration forces. FIG. 3 showed a set of three accelerometers 3-110, 3-120, 3-130 for the vertical, longitudinal and lateral directions, respectively. As is well known, force is acceleration multiplied by mass. In other words, force is directly derivable from acceleration in any of the three principal directions. FIG. 12 has been simplified by omitting the lateral direction.

In FIG. 12, the horizontal and vertical axes relate to longitudinal and vertical force, respectively. Reference number 12-11 denotes a vector having an optimal vertical-to-longitudinal force ratio. In the present context, an optimal vertical-to-longitudinal force ratio may depend on the parameter being optimized. Examples of the parameters to be optimized include running efficiency (minimization of energy consumption per distance), maximum velocity (minimum time per distance), or the like. Generally the vertical-to-longitudinal force ratio is optimal when the foot of the runner (test subject) detaches from the support at an angle of approximately 45°. This is because a freely moving body having a given start velocity travels a maximum distance if the direction of movement at the time of detachment is 45° with respect to horizontal. For each parameter to be optimized, the optimum angle may be determined empirically. Reference number 12-12 denotes a vector having an excessive vertical-to-longitudinal force ratio, while reference number 12-13 denotes a vector having a sub-optimal vertical-to-longitudinal force ratio.

FIG. 13 differs from FIG. 12 in that it illustrates calculation of actual and optimal ratios between brake force and vertical force at the time of strike (impact) on the support, such as ground. Brake force means the negative longitudinal force, which opposes the runner's motion at the time of impact. Reference numbers 13-11 and 13-12 denote two vectors of which the first has a markedly lower brake force compared with the second vector. In view of the fact that brake force is negative, or opposite to the direction of motion, the lower the brake force, the better. Similarly to FIG. 12, FIG. 13 has been simplified by omitting the lateral direction. While the lateral direction does not directly affect forward motion, it adds to the strain experienced by the runner's body and thereby increases energy consumption and risk of injury.

The calculated vertical-to-horizontal force ratios illustrated in FIGS. 12 and 13 can be indicated to the runner, preferably in real-time, so that the runner can learn the situations when the force ratios deviate from their optimum values. Real-time indication of the force ratios also permit the runner to try various corrective actions. A non-exhaustive list of corrective actions includes:
changing (typically increasing) cadence;
moving foot strike to fore foot;
reducing vertical movement; and
stiffening legs.

Various additional features may be combined with the above disclosure, in order to solve additional problems and/or to provide additional benefits. For instance, the inventive method may further comprise estimating the test subject's heart rate based on the test subject's running speed. The reception system may further receive heart rate sensor data from at least one heart rate sensor.

In some implementations, the method further comprises using the heart rate to determine at least one of the following parameters:
current oxygen intake value;
instantaneous energy expenditure;
running efficiency;
oxygen deficit;
excess post-exercise oxygen consumption.

The method may further comprise determining a gain value and a time constant for the test subject's current oxygen intake value and instantaneous energy expenditure. In some implementations the test subject's current oxygen intake value is determined based on the test subject′ heart rate, and wherein the test subject's instantaneous energy expenditure is determined based on the test subject's running speed.

The method may further comprise outputting the running efficiency versus time of day or versus exercise duration. Additionally or alternatively, the method may comprise outputting a body fatigue value versus exercise time.

In some implementations, the one or more dynamic movement parameters include one or more of:
vertical velocity and vertical position;
longitudinal velocity and longitudinal position;
lateral velocity and lateral position;
portions of time that the test subject is unsupported or supported by underlying ground;
stiffness of the test subject's legs;
upward propulsion energy and damping exerted by the test subject's legs;
forward propulsion energy exerted by the test subject's legs;
damping of the test subject's legs;
a ratio of vertical force to horizontal force at the time of detachment from and/or impact with the surface supporting the test subject's body.

In some implementations, said determining the instantaneous vertical, longitudinal and lateral acceleration may comprise compensating for gravity and orientation of the set of acceleration sensors.

In some implementations, the stored data further comprises at least one profile for each of several running styles, whereby an identification of running style and/or correction instructions may be outputted during an exercise.

In some implementations, the method further comprises using a set of inclination sensors to determine an inclination of the test subject's torso.

Those skilled in the art will realize that the inventive principle may be modified in various ways without departing from the scope of the present invention.

1. A method comprising:
performing a plurality of acts on a portable data processing system, wherein the portable processing system comprises:
a memory system for storing program code instructions and data, and a processing system for processing the data by executing the stored program code instructions;
a reception system for receiving data from a set of acceleration sensors, wherein the set of acceleration sensors is configured to indicate acceleration of the test subject in three orthogonal directions;
wherein the stored data comprises a plurality of conversion algorithms for converting measured parameters to processed parameters;
wherein the plurality of acts performed on the data processing system comprises:
receiving acceleration sensor data from the set of acceleration sensors;
determining instantaneous vertical acceleration, instantaneous longitudinal acceleration and instantaneous lateral acceleration from the received acceleration sensor data;
identifying periodicity in one of the instantaneous accelerations and using the identified periodicity with the instantaneous accelerations, to determine one or more dynamic movement parameters related to movement of the test subject's body over a surface supporting the test subject's body;
wherein one or more of the dynamic movement parameters comprise a first energy-related value indicative of vertical energy loss, a second energy-related value indicative of brake energy loss, which are determined separately for at least two legs of the test subject and for a combination of the legs;
using the first energy-related value and second energy-related value to determine a third energy-related value indicative of combined energy loss; and
outputting an indication of the combined energy loss and/or correction instructions.

2. The method according to claim 1, further comprising estimating the test subject's heart rate based on the test subject's running speed.

3. The method according to claim 1, wherein the reception system further receives heart rate sensor data from at least one heart rate sensor.

4. The method according to claim 2, wherein the method further comprises:
using the heart rate to determine at least one of the following parameters:
current oxygen intake value;
instantaneous energy expenditure;
running efficiency;
oxygen deficit;
excess post-exercise oxygen consumption.

5. The method according to claim 4, further comprising determining a gain value and a time constant for the test subject's current oxygen intake value and instantaneous energy expenditure.

6. The method according to claim 4, wherein the test subject's current oxygen intake value is determined based on the test subject's heart rate, and wherein the test subject's instantaneous energy expenditure is determined based on the test subject's running speed.

7. The method according to claim 4, further comprising outputting the running efficiency versus time of day.

8. The method according to claim 4, further comprising outputting the running efficiency versus exercise duration.

9. The method according to claim 1, further comprising outputting a body fatigue value versus exercise time.

10. The method according to claim 1, wherein the one or more dynamic movement parameters include one or more of:
vertical velocity and vertical position;
longitudinal velocity and longitudinal position;
lateral velocity and lateral position;
portions of time that the test subject is unsupported or supported by underlying ground;
stiffness of the test subject's legs;
upward propulsion energy and damping exerted by the test subject's legs;
forward propulsion energy exerted by the test subject's legs;
damping of the test subject's legs;
a ratio of vertical force to horizontal force at the time of detachment from and/or impact with the surface supporting the test subject's body.

11. The method according to claim 1, wherein said determining the instantaneous vertical, longitudinal and lateral acceleration comprises compensating for gravity and orientation of the set of acceleration sensors.

12. The method according to claim 1, further comprising using a set of inclination sensors to determine an inclination of the test subject's torso.

13. A portable data processing system, comprising:
a memory system for storing program code instructions and data,
a processing system for processing the data by executing the stored program code instructions;
a reception system for receiving data from a set of acceleration sensors, wherein the set of acceleration sensors is configured to indicate acceleration of the test subject in three orthogonal directions;
wherein the memory system comprises:
a plurality of conversion algorithms for converting measured parameters to processed parameters;
means for storing a profile for each of several running styles;
program code instructions for instructing the processing system to perform the acts cited in claim 1.

14. The portable data processing system according to claim 13, further comprising:
a set of inclination sensors, wherein the set of inclination sensors is configured to indicate orientation of the set of acceleration sensors;
wherein the set of inclination sensors is configured for mounting in a known orientation with respect to the test subject's torso.

15. A tangible memory carrier comprising program code instructions for a portable data processor, wherein execution of the program code instructions in the portable data processor causes the portable data processor to carry out the method according to claim 1.

16. The method according to claim 2, wherein said determining the instantaneous vertical, longitudinal and lateral acceleration comprises compensating for gravity and orientation of the set of acceleration sensors.

17. The method according to claim 3, wherein said determining the instantaneous vertical, longitudinal and lateral acceleration comprises compensating for gravity and orientation of the set of acceleration sensors.

18. The method according to claim 4, wherein said determining the instantaneous vertical, longitudinal and lateral acceleration comprises compensating for gravity and orientation of the set of acceleration sensors.

19. The method according to claim 5, wherein said determining the instantaneous vertical, longitudinal and lateral acceleration comprises compensating for gravity and orientation of the set of acceleration sensors.

20. The method according to claim 6, wherein said determining the instantaneous vertical, longitudinal and lateral acceleration comprises compensating for gravity and orientation of the set of acceleration sensors.
